(19) United States
(12) Patent Application Publication
(10) Pub. No.: US 2010/0137592 A1
(43) Pub. Date: Jun. 3, 2010

(54) PROCESS FOR PREPARING PURINE DERIVATIVE

(76) Inventors: Asif Parvez Sayyed, Hyderabad (IN); Murali Krishna Ankaraju, Hyderabad (IN); Ravinder Reddy Vennapureddy, Hyderabad (IN); Shankar Rama, Hyderabad (IN); Ramesh Dandala, Hyderabad (IN); Sivakumaran Meenakshisunderam, Hyderabad (IN)

Correspondence Address: Jay R Akhave, 2058 N Mills Ave, #612, Claremont, CA, 91711, US

(21) Appl. No.: 12/452,196

(22) PCT Filed: Jun. 2, 2008
(86) PCT No.: PCT/IB2008/001481

§ 371 Date: Dec. 19, 2009

(30) Foreign Application Priority Data
Jun. 21, 2007 (IN) 1287/CHE/2007

(51) Int. Cl. C07D 473/32; C07D 239/50; C07C 255/37; C07C 217/48; C07C 213/02; C07C 213/08

(52) U.S. Cl. 544/277; 544/323; 558/389; 564/346; 564/490; 564/493

(57) ABSTRACT

A process for the preparation of famciclovir a compound of Formula (I) and its intermediates.

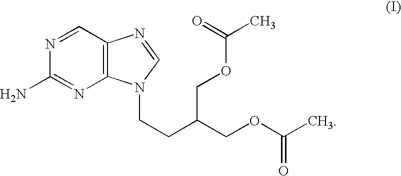

### FIELD OF THE INVENTION

The invention relates to a novel process for the preparation of famciclovir and its intermediates.

### BACKGROUND OF THE INVENTION

9[4-acetoxy-3-(acetoxymethyl)but-1-yl]-2-aminopurine of Formula I,

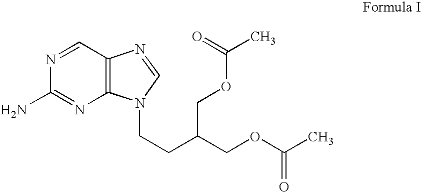

is generically known as Famciclovir, a drug of purine derivative having antiviral activity. Famciclovir is being marketed under the Trade name FAMVIR as tablet. Famciclovir for the first time was disclosed in U.S. Pat. No. 5,075,445.

In view of the importance of famciclovir as a antiviral drug, several synthetic methods have been reported in the literature to prepare famciclovir, which are as summarized below:

A number of different routes for preparation of famciclovir are known including those described in EP 0 182 024 B1, U.S. Pat. No. 5,684,153, U.S. Pat. No. 5,138,057, U.S. Pat. No. 6,761,767. A general approach to prepare famciclovir is as summarized below:

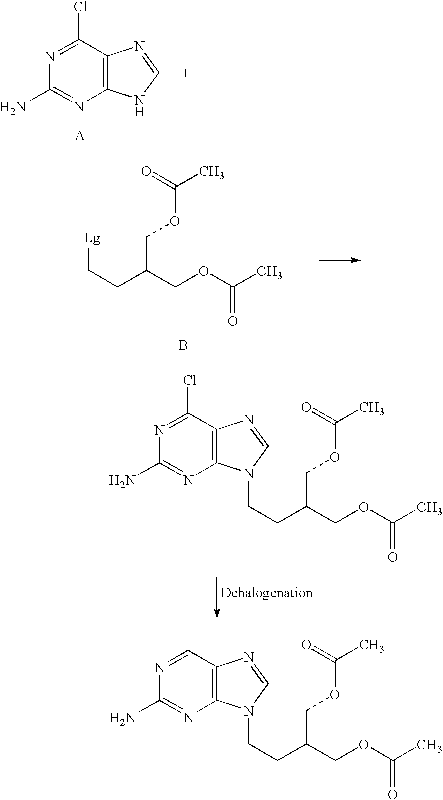

wherein Lg represents Cl, Br, I or any other leaving group.

The process to prepare intermediate B is as summarized below:

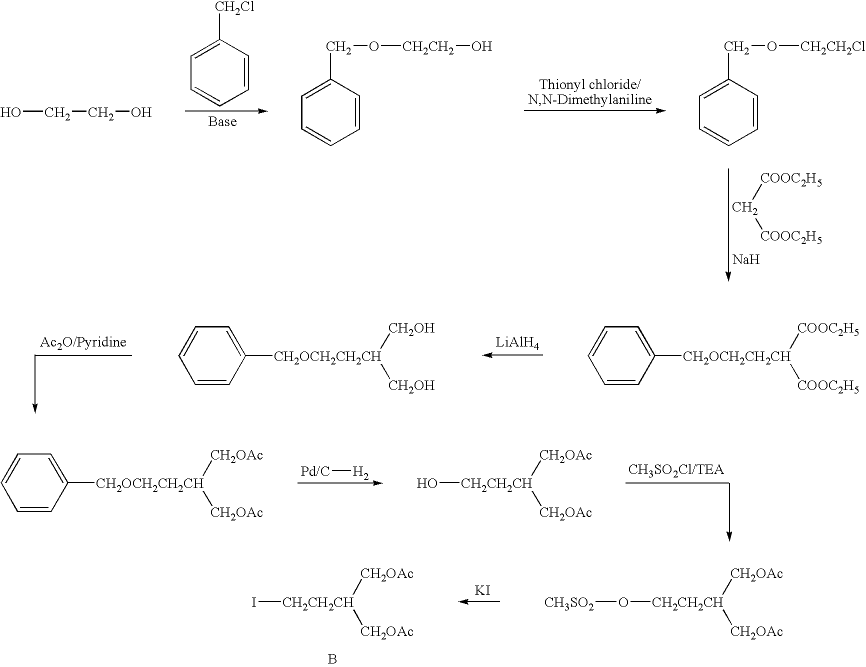

Famciclovir prepared by the above process has a common problem that is lack of regioselectivity during the N-alkylation reaction, as the undesired 7-position isomer is generated simultaneously reducing the yields. Further it requires a separate purification step to remove the unwanted isomer. The intermediate compound B is prepared by a lengthy procedure, which involves a number of synthetic steps and uses reagents, which are difficult to handle and the overall yield is low.

There have been a number of attempts to increase the regioselectivity of this N-alkylation reaction e.g. by having different substituents at the 6^{th} position of purine moiety, which are known to affect the N−7/N−9 ratio or by using 2-amino-6,8-dichloro purine as substrate or using a purine derivative where the N−7 position is already blocked. Another approach to achieve the regioselectivity involved reacting 2-amino-6-chloropurine with an allyl derivative in presence of a palladium (0) catalyst and a suitable ligand followed by effecting a rearrangement of N−7 alkylated purine derivative to the N−9 alkylated analogues. However all the above approaches involve costly raw materials, increased number of synthetic steps resulting in overall low yield. Also in most of the cases, the desired starting compounds are not commercially available and have to be prepared separately, making its industrial application further difficult.

An alternate approach which provides a solution to the above mentioned regioselectivity problem lies in introducing the alkyl side chain first into the desired position of the pyrimidine to get substituted pyrimidine derivative, followed by ring closure to get the N−9 substituted purine base [U.S. Pat. No. 5,971,041, WO 2004/110343]. The process of WO 2004/110343 A2 is lengthy and involves ten synthetic steps starting from 5-Nitro uracil and gives very low overall yield of Famciclovir.

In U.S. Pat. No. 5,971,041 a synthetic route for famciclovir is reported where N-(2-amino-4,6-dichloro-5-pyrimidinyl) formamide is reacted with 2-acetoxymethyl-4-aminobut-1-yl-acetate in presence of a base. However, we found that in the experimental conditions described for this reaction, the side chain 2-acetoxy-methyl-4-amino-butyl-1-yl-acetate gets cyclised in presence of base to give (pyrrolidin-3-yl)methyl acetate of the following formula.

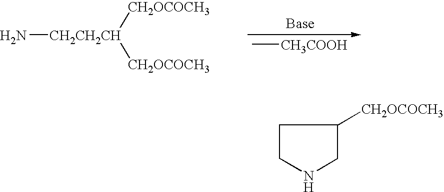

Synthetic communications 2(6), 345-351 (1972, discloses a process to prepare chloropurine/hypoxanthine analogs. The process is as shown below:

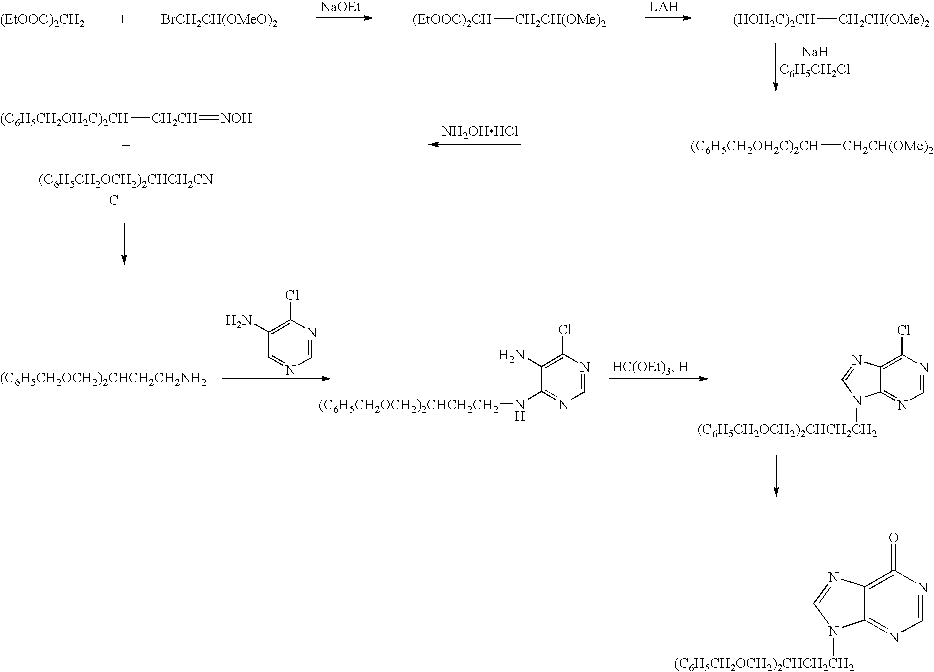

This publication does not disclose any process for the preparation of famciclovir. Further, the intermediate 3,3-bis(benzyloxymethyl)propionitrile of Formula C has been obtained as a mixture with the oximes (syn & anti).

### OBJECTIVE

The object of the present invention is to provide new intermediates, which can be effectively used in the preparation of famciclovir.

Yet another object of the present invention is to provide an improved process for the preparation of famciclovir, which has high selectivity leading to the improved process efficiency, with a reduced number of steps, and improved yield.

Yet another object of the present invention is to provide a method for the preparation of famciclovir, which allows the use of cheaper and easy to handle reagents and applying milder reaction conditions.

### SUMMARY OF THE INVENTION

The present invention relates to a process for preparing a compound of Formula II,

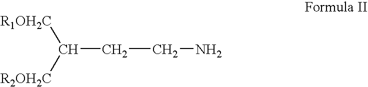

wherein R₁ and R₂ represents hydroxy protecting group
which comprises:
i) reacting a compound of Formula III

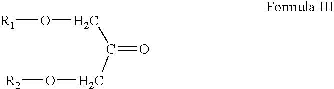

wherein R₁ and R₂ is same as defined above
with a compound of Formula IV

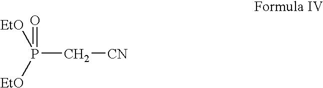

in the presence of a base and a solvent to give a compound of Formula V

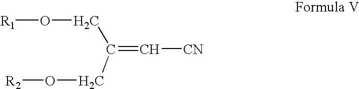

wherein R₁ and R₂ is same as defined above
ii) reducing a compound of Formula V to give a compound of Formula II.

In another embodiment of the present invention, the compound of Formula II is converted to famciclovir and its pharmaceutically acceptable salts thereof.

### DETAILED DESCRIPTION OF THE INVENTION

Hydroxy protecting group is selected from the group C₆H₅CH₂—,

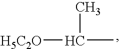

acetonide, acetal of 1,3-dioxane such as

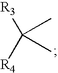

R₃ and R₄ is selected from hydrogen, C₁₋₅ alkyl, C₃₋₈ cycloalkyl, substituted or unsubstituted aryl.

The compound of Formula III,

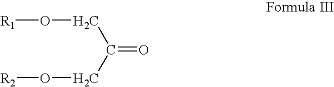

wherein R₁ and R₂ represents hydroxy protecting group, is reacted with a cyano compound of Formula IV to give a compound of Formula V in the presence of base and a solvent. The base is selected from sodium hydride, n-butyl lithium, potassium carbonate and the solvent is selected from toluene, tetrahydrofuran, 1,1-dimethoxyethane, methylene chloride, benzene, preferably toluene or tetrahydrofuran. The compound of Formula V is reduced to give 4-amino-2-hydroxymethyl-1-butanol of Formula II. The reduction is carried out using lithium aluminium hydride or by catalytic hydrogenation using palladium/carbon, Raney nickel in a solvent selected from methanol, ethanol, isopropanol.

The compound of Formula III is prepared using a process disclosed in US 2002/0193324 by reacting a compound of Formula VI,

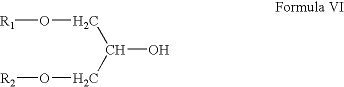

with an oxidizing agent in the presence of a solvent and TEMPO as a catalyst. But the process disclosed in US 2002/0193324 was slightly modified. The modification was in the form of adding dilute hydrochloric acid during the oxidation reaction to maintain the pH in the range of 9.0±0.2, as it was observed that the reaction at higher pH did not proceed. In order to make the process cost effective and commercially viable, the quantities of acetonitrile and TEMPO has been drastically reduced without affecting the yield and quality of the product. In fact the yield obtained in the modified process is almost quantitative with product purity exceeding 98% (by GC).

The compound of Formula VI is prepared by the process disclosed in Journal of Medicinal Chemistry 1993, 26, 759-61.

The compound of Formula V is reduced in one step in to compound of Formula II using an reducing agent in the presence or absence of an acid such as acetic acid. Reducing agent is selected from Lithium aluminium hydride or by catalytic hydrogenation using a noble metal catalyst such as Pd/C, Pt/C, Raney nickel, Rhodium, Platinum oxide. When Lithium aluminium hydride is used the solvent is selected from tetrahydrofuran or ether.

Further, above conversion can also be carried out in step wise in an organic solvent. The stepwise reduction is preferred. The suitable solvent is selected ethanol, methanol and isopropanol, preferably methanol. The reduction steps can be carried out in presence or absence of an acid. The reduction is carried by catalytic hydrogenation using a noble metal catalyst such as Pd/C, Pt/C, Raney nickel, Rhodium, Platinum oxide.

The compound of Formula V

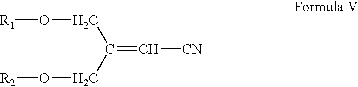

is first reduced to a compound of Formula VII

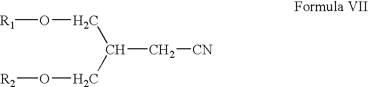

and then reduced to amine of compound of Formula II

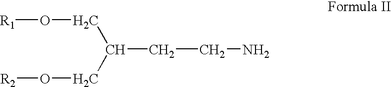

in presence of ammonia to avoid the formation of secondary amine byproduct

The compound of Formula II (when R represents C₆H₅CH₂—) is catalytically hydrogenated to give a compound of Formula VIII

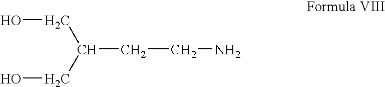

in the presence of a solvent. Catalytic hydrogenation is carried out using a noble metal catalyst such as Pd/C, Pt/C, Raney nickel, Rhodium, Platinum oxide and the solvent is methanol.

The compound of Formula II (when R represents

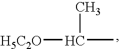

acetonide, acetal of 1,3-dioxane such as

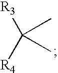

R₃ and R₄ is selected from hydrogen, C₁₋₅ alkyl, C₃₋₈ cycloalkyl, substituted or unsubstituted aryl) is converted to a compound of Formula VIII

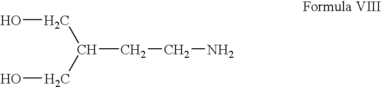

in the presence of a dilute acid. Acid is selected from hydrochloric acid, sulfuric acid, nitric acid, preferably hydrochloric acid.

The compound of formula II is used as an intermediate in the preparation of famciclovir of compound of Formula I, which is as shown below:

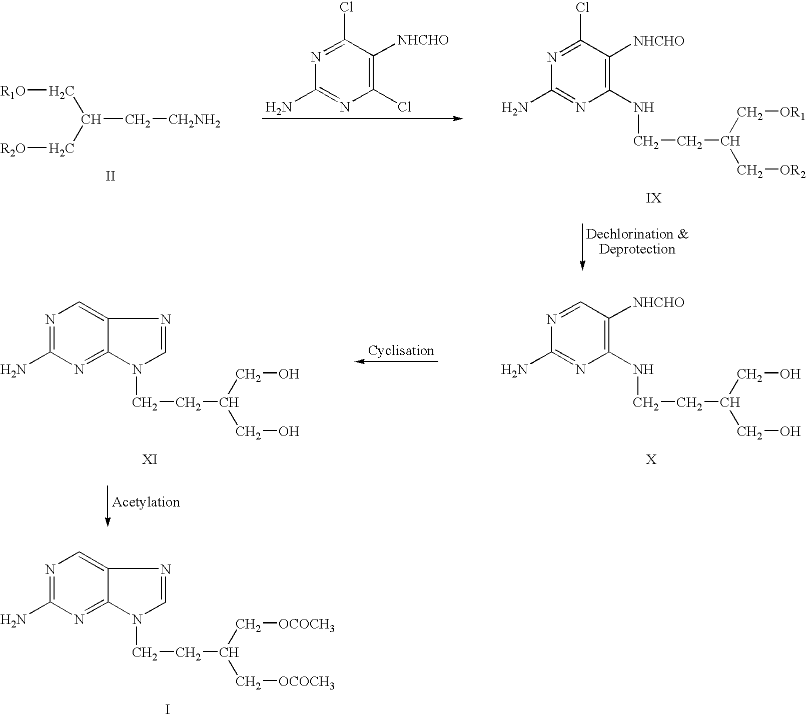

wherein R₁ and R₂ represents hydroxy protecting group

The compound of Formula II is reacted with N-(2-amino-4,6-dichloro-5-pyrimidinyl) formamide in a solvent and a base to give a compound of Formula IX in a solvent selected from ethanol, isopropanol, n-butanol, dimethyl formamide, dimethylsulfoxide, acetonitrile, methylisobutylketone, toluene and mixtures thereof, preferably ethanol, isopropanol and base selected from potassium carbonate, potassium bicarbonate, sodium bicarbonate, sodium carbonate, sodium ethoxide, sodium methoxide, triethylamine, preferably sodium bicarbonate.

The compound of Formula IX is isolated or insitu converted to compound of Formula X.

The compound of Formula IX is dechlorinated and deprotected to give a compound of Formula X using catalytic hydrogenation in presence of noble metal catalyst such as Pd/C. The dechlorination reaction can be done either in presence or absence of a base, however the dechlorination in presence of base, which quenches the byproduct hydrochloric acid is preferred. The base is selected from inorganic or organic base such as triethylamine. The reaction can be done at room temperature or at reflux temperature of the solvent.

The compound of Formula X is cyclized in presence of acid catalyst using orthoformate ester such as triethyl orthoformate, trimethyl orthoformate or diethoxymethyl acetate etc., at a temperature of 25° C.-150° C., more preferably at 45-55° C. Acid catalyst is selected from formic acid, gaseous hydrogen chloride, aqueous hydrochloric acid, sulfuric acid. The moisture content of solution (of compound of formula X) taken for cyclisation can be in the range of 0-5%, preferably in the range of 1-3%. The cyclised product is acetylated to get famciclovir or it can be crystallized from a solvent selected from aqueous ethanol or aqueous methanol before proceeding for acetylation reaction.

The compound of Formula VIII is used an intermediate in the preparation of famciclovir as disclosed in our co-pending application No. IN 2291/CHE/2006.

The present invention also relates to novel intermediate of compound of Formula V

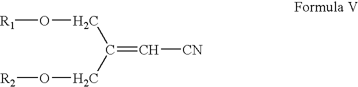

wherein R₁ and R₂ represents hydroxy protecting group.

The present invention also relates to novel intermediate of compound of Formula IX

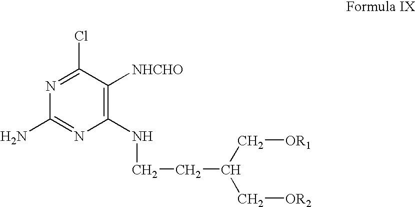

wherein R₁ and R₂ represents hydroxy protecting group.

Diethyl cyanomethyl phosphonate of Formula IV, was prepared by the Michaelis-Arbuzov reaction utilizing triethyl phosphite and chloroacetonitrile.

1,3-Bis(benzyloxy)acetone is treated with diethylcyanomethyl phosphonate in the presence of sodium hydride and toluene to obtain a 3,3-bis (benzyloxymethyl)acrylonitrile. The 3,3-bis(benzyloxymethyl)acrylonitrile compound is reduced using Pd/C (double-bond reduction) and then reduced using Raney nickel (Cyano group reduction) in methanolic ammonia to give 3,3-bis(benzyloxymethyl)propionitrile. Debenzylating the 3,3-bis(benzyloxymethyl)propionitrile compound using Pd/C to give 4-amino-2-hydroxymethyl-1-butanol of Formula VIII.

The intermediate 3,3-bis(benzyloxymethyl)propionitrile has been earlier obtained in Synthetic Communications 1972, 2(6), 345-351 as a mixture with the oximes (syn & anti) when 3,3-bis(benzyloxymethyl) propionaldehyde diethyl acetal was refluxed with hydroxylamine hydrochloride in methanol. The mixture was further reduced using lithium aluminium hydride to get the corresponding amine. However in the present process the 3,3-bis(benzyloxymethyl)propionitrile has been prepared as single product having GC purity ≧98%, which is subsequently reduced to give amine.

The invention includes the method for effectively preparing famciclovir using the above intermediates, which shows 100% selectivity, so that 7-[4-acetoxy-3-(acetoxymethyl)but-1-yl]-2-aminopurine which is pharmaceutically inactive isomer of famciclovir is not produced at all as a byproduct.

The invention is illustrated with the following examples, which are provided by way of illustration only and should not be construed to limit the scope of the invention.

### Example 1

### Preparation of 1,3-di-o-benzylglycerol

A solution of sodium hydroxide (99.5 g) in water (95 ml) was added to benzyl alcohol (362 g) at 25-45° C. in 10-15 min. Epichlorohydrin (100 g) was added to reaction mixture dropwise while stirring over 30-40 min at 25-45° C. The reaction mass was stirred for ˜22 h at 25-35° C., where upon epichlorohydrin reacts completely and the mono benzyloxy derivative was <3.0%. The reaction mass was diluted with water (500 ml) and toluene (500 ml) was added and stirred for 5-10 min. The layers were separated and the organic layer was evaporated to dryness under reduced pressure to give an oily residue (˜400 g), which was subjected to fractional distillation. The first fraction distilling at 115-175° C. (2-5 mm Hg) contained mainly unreacted benzyl alcohol and the monobenzyloxy derivative. The main fraction (220 g) distilling at 185-215° C. was collected to give title compound of GC purity >99.5%.

### Example 2

### Preparation of 1,3-dibenzyloxy-2-propanone

To a solution of 1,3-Di-O-benzylglycerol (100 g) in acetonitrile (400 ml), 2,2,6,6-Tetramethyl-1-piperidinyloxy free radical (TEMPO, 1.1 g) was added under nitrogen atmosphere. To the reaction solution sodium bicarbonate (31 g) dissolved in water (320 ml) was added. The solution was cooled to 1-3° C. and sodium hypochlorite solution (375 g, assay 9.5% w/w=35.6 g on 100% basis) was added slowly over ˜90 min at 1-7° C. while maintaining pH of reaction mass throughout addition at 9.0±0.2 by adding dilute hydrochloric acid. The reaction mass was stirred at 1-7° C. for 30 minutes for complete disappearance of 1,3-di-O-benzyl glycerol by TLC/GC. Reaction mass was diluted with water (380 ml) and product was extracted with methylene dichloride (1×400 ml, 1×100 ml). The combined organic extract was stirred with 15% w/v sodium sulphite solution (150 ml) for 20 min at 10-15° C. Thereafter, the methylene dichloride solution was washed with saturated sodium chloride solution (200 ml) and evaporated under reduced pressure at 25-30° C. to an oily residue. To the oily residue toluene (100 ml) was added and distilled completely under reduced pressure to remove residual acetonitrile/MDC. Title product was obtained as orange colour oil which solidifies on standing.

Yield: 98.3 g (contains-2% toluene)

GC Purity ≧98.5%.

### Example 3

### Preparation of 3,3-bis(benzyloxymethyl)acrylonitrile

To a cooled suspension of sodium hydride (14.8 g, 60% w/w) in toluene (600 ml) under nitrogen atmosphere diethyl cyanomethyl phosphonate (65.5 g) was added at 3-10° C. slowly in 60-70 min. After completion of addition, a thick slurry results. Thereafter, the slurry temperature was raised to 15-20° C. and stirred for 30 min. The slurry was again cooled to 3-5° C. and a solution of 1,3-dibenzyloxy-2-propanone (100 g) in toluene (200 ml) was added over 30-40 min at 3-8° C. Reaction mass was stirred at 3-8° C. and monitored by TLC/GC. The reaction was complete in approximately 30 min. To the reaction mass precooled ethanol (160 ml, 0-2° C.) was added at 3-18° C. and stirred for 25-30 min to obtain a clear solution. Thereafter, precooled water (600 ml, 5-10° C.) was added at 12-18° C. and continued stirring at 12-18° C. for 20 min. Toluene layer was separated and washed with water (200 ml) and dried over anhydrous sodium sulphate, filtered and evaporated under reduced pressure to give an oily residue containing the title compound.

Yield: 106.2 g (contains ˜0.8% toluene, 97% of theory, GC purity ≧98.3%)

IR (neat): (ν) 3089 m, 3065 m, 3032 m, 2863 s, 2222 s (—CN), 1641 m, 1497 s, 1470 m, 1455 s, 1390 m, 1368 s, 1311 w, 1295 w, 1275 m, 1254 m, 1208 m, 1177 m, 1102 s, 1029 s, 1002 s, 945 m,

¹H NMR (300 MHz, CDCl₃): 7.31-7.39 (m, 10H, Phenyl), 5.71 (s, 1H, CH, vinyl), 4.56 (s, 2H, CH₂-Phenyl), 4.51 (s, 2H, CH₂-Phenyl), 4.33 (s, 2H, CH₂O), 4.21 (s, 2H, CH₂O)

¹³C NMR (CDCl₃): δ8.9, (s, CH₂O), 69.8 (s, CH₂O), 73.6 (s, CH₂-Phenyl), 96.7 (s, CH vinyl), 116.5 (s, CN), 128.1-129.6 (m, CH, Phenyl), 137.9 (s, C, Phenyl), 138.0 (s, C, Phenyl), 160.7 (s, C, vinyl). [M+H]⁺=294, [M+NH₄]⁺=311.3, [M+Na]⁺=316.3

### Example 4

### Preparation of 3,3-bis(benzyloxymethyl)propionitrile

To a solution of 3,3-Bis(benzyloxymethyl)acrylonitrile (100 g) in methanol (600 ml) 5% palladium on charcoal (3 g, ˜50% wet) was added and hydrogenated at 30 psi hydrogen pressure for 2 h at 20-30° C. The catalyst was filtered and the filtrate was evaporated under reduced pressure to obtain 3,3-bis(benzyloxymethyl) propionitrile as an oil.

Yield: 98.2 g

GC Purity ≧98%,

¹H NMR (300 MHz, CDCl₃): 2.31-2.37 (m, 1H, CH), 2.56-259 (2H, CH₂CN), 3.47-3.61 (m, 4H, CH₂O), 4.51 (s, 4H, CH₂—Ar), 7.26-7.37 (m, 10H, Aromatic), [M+H]⁺=296.3, [M+NH₄]⁺=313.3, [M+Na]⁺=318.2

### Example 5

### Preparation 3,3-bis(benzyloxymethyl)propylamine

To a solution of 3,3-Bis(benzyloxymethyl)propionitrile (98.2 g, as obtained in example 4) in methanolic ammonia (590 ml, 12% w/w), Raney Nickel (30 ml, ˜18 g, prewashed with water/methanol to remove alkali) was added and the slurry was hydrogenated at 7-8 Kg hydrogen pressure for 4 h at 20-35° C. The catalyst was filtered off and the filtrate was evaporated under reduced pressure (˜50 mm Hg) to dryness to obtain the title compound as light greenish oil

Yield: 99.5 g, 98% of theory,

GC Purity ≧98.2%

¹HNMR (CD₃OD+a drop of TFA, 300 MHz): 1.89-1.96 (m, 2H, CH₂, CH—CH₂), 2.15-2.19 (m, 1H, CH), 3.17 (m, 2H, CH₂, CH₂NH₂), 3.51-3.69 (m, 4H, CH₂O), 4.59 (s, 4H, CH₂ of benzyl), 7.40-7.46 (m, 10H, Aromatic). [M+H]⁺=300.3

### Example 6

### Preparation of 3,3-bis(benzyloxymethyl)propylamine

To the solution obtained after catalyst filtration in example 4 [containing 3,3-Bis(benzyloxymethyl)proprionitrile] ammonia gas was bubbled till the content of ammonia in solution reached to 10+1% w/w. To the solution Raney nickel (30 ml, ˜18 g) was added. Hydrogenation and workup was carried out as in example 5 to obtain title compound.

Yield: 100.3 g.

GC Purity ≧98.2%

### Example 7

### Preparation of 4-amino-2-hydroxymethyl-1-butanol hydrochloride

To a solution of 3,3-bis(benzyloxymethyl)propylamine (99 g, in methanol (590 ml) concentrated hydrochloric acid (34.5 g, 35% w/w) and 5% palladium on charcoal (8 g, ˜50% wet) were added and hydrogenated at 7-8 Kg/cm² hydrogen pressure for 5 h at 20-35° C. The catalyst was filtered off and the filtrate was evaporated under reduced pressure to obtain 4-amino-2-hydroxymethyl-1-butanol hydrochloride as greenish oil.

Yield: 51.5 g (quantitative, Purity ≧97%)

¹H NMR (300 MHz, DMSO-d₆): 1.57 (m, 3H, CH—CH₂—CH₂—NH₂); 2.83 (m, 2H, CH₂NH₂); 3.29-3.41 (m, 4H, OCH₂). [M+H]⁺=120, [M+Na]⁺=142

### Example 8

### Preparation of 4-amino-2-hydroxymethyl-1-butanol

To a stirred solution of 3,3-bis(benzyloxymethyl)acrylonitrile (100 g) in acetic acid (650 ml), 5% palladium on charcoal (15 g, ˜50% wet) was added and hydrogenated at 2 Kg/cm² hydrogen pressure for 2 h at 25-35° C. Thereafter, the reaction mass was heated to 60-65° C. and hydrogenation continued at 8.0 Kg/cm² hydrogen pressure till the hydrogen consumption has stopped. The catalyst was filtered off, evaporated acetic acid under reduced pressure to obtain 4-Amino-2-hydroxymethyl-1-butanol acetate as greenish oil. The residue was dissolved in water and purified by passing through an ion exchange resin to obtain 4-amino-2-hydroxymethyl-1-butanol.

Yield: 26 g, 64% of theory

### Example 9

### Preparation of 4-amino-2-hydroxymethyl-1-butanol hydrochloride

Lithium aluminium hydride (3.22 g) was added in 15-20 minutes to anhydrous tetrahydrofuran (40 ml) at 23-28° C. and the reaction suspension was cooled to 0-5° C. under nitrogen atmosphere. A solution of 3,3-bis(benzyloxymethyl)acrylonitrile (15 g) in tetrahydrofuran (20 ml) was added to Lithium aluminium hydride at 0-5° C. in approximately 30 min. The reaction mixture was stirred for further 1 h and the reaction was quenched by slow addition of saturated sodium sulfate solution at 0-5° C. The salts were filtered and washed the residue with tetrahydrofuran (20 ml). The filtrate was evaporated to dryness under reduced pressure to obtain 3,3-bis(benzyloxymethyl)propylamine as oily liquid (15 g). This oily residue (15 g) was dissolved in methanol (90 ml) and treated with activated carbon (1.5 g) and filtered to obtain clear filtrate. Conc. hydrochloric acid (5.20 g, 35% w/w) and 5% Pd/C (1.2 g, ˜50% wet) were added to the clear filtrate and debenzylation reaction and workup were carried out as described in example 7 to give title compound as greenish oil

Yield: 5.70 g, 71.6% of theory,

Purity: 86.2%

### Example 10

### Preparation of 2-amino-9-(4-hydroxy-3-hydroxymethyl but-1-yl)purine hydrochloride

To a suspension of N-(2-amino-4,6-dichloro-5-pyrimidinyl)formamide (100 g) in ethanol (600 ml), powdered sodium bicarbonate (121.8 g) was added and heated to mild reflux at 76-82° C. An aqueous solution of 4-amino-2-hydroxymethyl-butan-1-ol hydrochloride (60% w/w, 140 g eq. to 84 g on 100% basis) was diluted with ethanol (300 ml) and added slowly over a period of 60 min to reaction mass maintaining a mild reflux (74-82° C.). Refluxing was continued for 90 min and reaction was monitored by HPLC where the unreacted formamido compound was observed to be <2.0%. Reaction mass was cooled to 18-22° C. and filtered, to remove inorganics and residue was washed with hot ethanol (150 ml, approximately 50° C.). The orange red coloured solution was treated with activated carbon (3 gm) and refiltered. Triethylamine (62 g) and 10% Pd/C (12 g, approximately 50% wet) were added to the filtrate. This solution was transferred to an autoclave and hydrogenated at 8-9 Kg hydrogen pressure at 55-60° C. for 24 h to complete the dichlorination reaction. Catalyst was filtered and washed the residue with ethanol (100 ml). The clear filtrate (approximately 1200 ml) was concentrated under reduced pressure to approximately 300 ml volume. Fresh ethanol (approximately 235 ml) was added to achieve moisture content of the solution 2.3% w/w and also to get the total volume of reaction mass 500 ml. Hydrogen chloride solution in ethanol (approximately 21% w/w, 130 g) and triethyl orthoformate (340 g) were added to the solution at 20-30° C. Thereafter, the reaction mass was heated to 50-55° C. and stirred for 3 h at 50-55° C. Thereafter a mixture of 36 ml water and 12 g conc. hydrochloric acid was added over a period of 5-10 min at 50-55° C. and stirring continued further for two hours at 50-55° C. After approximately 20 min of adding aqueous hydrochloric acid, product starts crystallizing out. The slurry was cooled to 20-30° C. and maintained for 3 h. Product was filtered and washed with ethanol (100 ml, 30° C.). Product was dried for 3 h at 60-70° C. till constant weight was achieved.

Yield: 100.6 g (76% of theory),

Chromatographic Purity (HPLC): ≧97.0%

The above obtained product can be taken as such for famciclovir preparation in next stage of or could be further purified by the following procedure.

The above obtained product (100.4 g) was suspended in a mixture of ethanol (635 ml) and water (30 ml) and heated and refluxed for 30 min. Thereafter approximately 200 ml aqueous ethanol was distilled from the slurry at 78-81° C. under atmospheric pressure. The product slurry was cooled to 25-30° C., filtered and washed with ethanol (90 ml). Product was dried at 60-70° C. till constant weight was achieved.

Yield: 88.7 g (88.3% recovery)

Chromatographic Purity (HPLC)≧99.2%

¹H NMR (300 MHz, DMSO-d₆): 1.47 (m, 1H, CH), 1.81 (m, 2H, NCH₂CH₂—CH), 3.3-3.47 (m, 4H, CH₂OH), 4.18 (t, NCH₂), 8.12 (s, 2H, NH₂), 8.66 (s, 1H, H-8 of purine), 9.00 (s, 1H, H-6 of purine) [M+H]⁺=238

### Example 11

### Preparation of 9-[4-acetoxy-3-(acetoxymethyl)but-1-yl]-2-aminopurine (famciclovir)

2-Amino-9-(4-hydroxy-3-hydroxymethylbut-1-yl)purine Hydrochloride (75 g) was suspended in methylene dichloride (300 ml) and cooled to 10-20° C. Triethylamine (91 g) diluted with methylene dichloride (50 ml) was added over a period of 15-20 min. 4-(dimethylamino pyridine) (DMAP, 0.5 g) was added at 10-20° C. and the slurry was cooled to 5-8° C. Acetic anhydride (65 g) diluted with methylene dichloride (200 ml) was added slowly over 60 min at 5-10° C. Reaction mass was stirred for 2 h at 5-10 and hydrogen chloride solution in ethanol (15 ml) was added. The solution was stirred for 10 min and methylene dichloride was evaporated under reduced pressure at 30-35° C. To the residue water (375 ml) was added and distilled to remove residual methylene dichloride. The aqueous solution was treated with activated carbon (4 g). The clear filtrate was cooled to 10-15° C. and seeded with famciclovir (0.10 g). Stirring was continued for 6 h to complete the crystallization of product. The product slurry was cooled to 5-10° C. and maintained for 2 h. Product was filtered and washed with chilled water (70 ml, 4-6° C.) and dried under reduced pressure at approximately at 50° C. till constant weight to give title compound.

Yield: 73 g

Chromatographic purity (HPLC): ≧99.3%

The above obtained product (73 g) was dissolved was dissolved in ethyl acetate (440 ml) at 50-60° C. and filtered through hyflo. The clear filtrate was concentrated under vacuum at 55-60° C. to distill out ethyl acetate (approximately 250 ml). To the concentrate, hexane (270 ml) was added at 40-45° C. and slurry was cooled to 5-8° C. It was stirred at 5-8° C. for 60 min and filtered. The product obtained was washed with chilled hexane (65 ml) and dried till constant weight.

Yield: 66.5 g (91%)

Chromatographic purity (HPLC)≧99.8%

Mono hydroxy analog: <0.15%

Mono propionyl analog (FW 335)<0.10%

Dihydroxy Famciclovir <0.05%

N-acetyl Famciclovir: <0.10%

We claim:

1) A process for preparing a compound of Formula II, 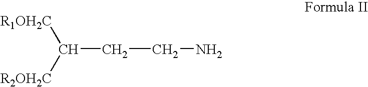
wherein R₁ and R₂ represents hydroxy protecting group selected from the group C₆H₅CH₂—, 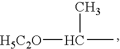
acetonide, acetal of 1,3-dioxane such as 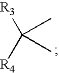
R₃ and R₄ is selected from hydrogen, C₁₋₅ alkyl, C₃₋₈ cycloalkyl, substituted or unsubstituted aryl
which comprises:
i) reacting a compound of Formula III 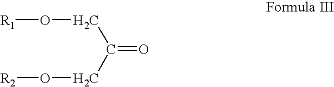
wherein R₁ and R₂ is same as defined above
with a compound of Formula IV 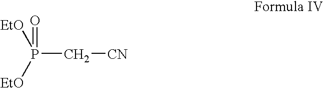
in the presence of a base and a solvent to give a compound of Formula V 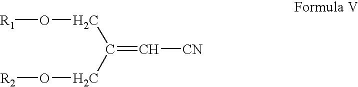
wherein R₁ and R₂ is same as defined above
ii) reducing a compound of Formula V to give a compound of Formula II.

2) The process according to claim 1, wherein the base in step i is selected from sodium hydride, n-butyl lithium, potassium carbonate and solvent is selected from toluene, tetrahydrofuran, 1,1-dimethoxyethane, methylene chloride, benzene, preferably toluene or tetrahydrofuran

3) The process according to claim 1, wherein the reduction in step ii is carried out using lithium aluminium borohydride or the catalytic hydrogenation using a noble catalyst palladium/carbon, Platinum/carbon, Raney nickel.

4) The process according to claim 1, the reduction is carried out in solvent selected from methanol, ethanol, isopropanol, tetrahydrofuran, ether.

5) The process according to claim 3, the reduction is carried out in one step or step wise in the presence or absence of an acid such as acetic acid.

6) The process according to claim 1, the compound of Formula II is reduced using catalytic hydrogenation in the presence of a noble catalyst selected from palladium/carbon, Platinum/carbon, Raney nickel, Rhodium, Platinum oxide to give a compound of Formula VIII

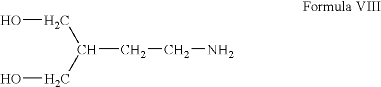

7) (canceled)

8) A compound of Formula V 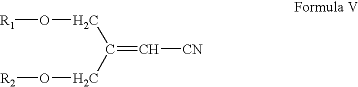
wherein R₁ and R₂ represents hydroxy protecting group selected from the group C₆H₅CH₂—, 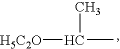
acetonide, acetal of 1,3-dioxane such as 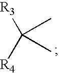
R₃ and R₄ is selected from hydrogen, C₁₋₅ alkyl, C₃₋₈ cycloalkyl, substituted or unsubstituted aryl.

9) A compound of Formula IX 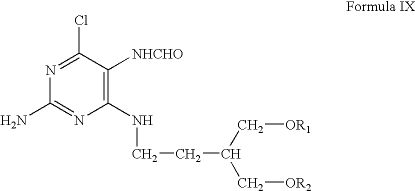
wherein R₁ and R₂ represents hydroxy protecting group selected from the group C₆H₅CH₂—, 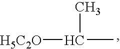
acetonide, acetal of 1,3-dioxane such as 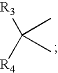
R₃ and R₄ is selected from hydrogen, C₁₋₅ alkyl, C₃₋₈ cycloalkyl, substituted or unsubstituted aryl.

10) The compound 3,3-bis(benzyloxymethyl)propionitrile of Formula C isolated with a purity greater than 98% as measured by Gas Chromatography.

11) A compound of Formula X

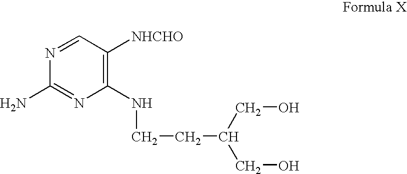

12) The process according to claim 1, the compound of Formula II is converted to give a compound of Formula VIII 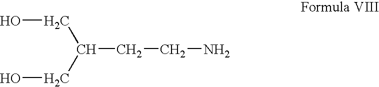
in the presence of a dilute acid.

13) The process according to claim 12, wherein the acid is selected from hydrochloric acid, sulfuric acid, nitric acid.

14) A process for the preparation of Famciclovir of Formula I 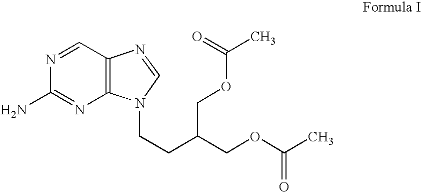
which comprises
a) condensing the compound of Formula II, 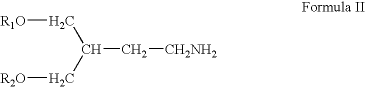
wherein R₁ and R₂ is same as defined above
with a compound of Formula XII, 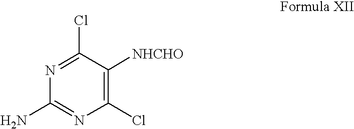
in the presence of base in a solvent to give compound of Formula IX, 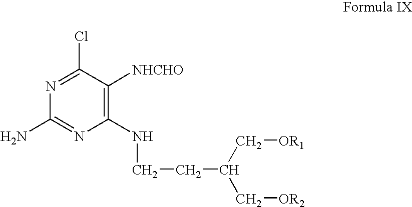
b) dechlorinating and deprotection of the compound of Formula IX using a catalyst in a solvent to obtain a compound of Formula X, 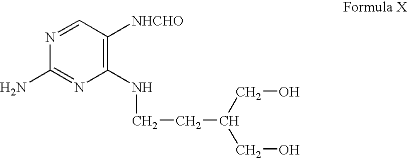
c) cyclizing the compound of Formula X with trialkylorthoformate in the presence of an acid to give compound of Formula XI, 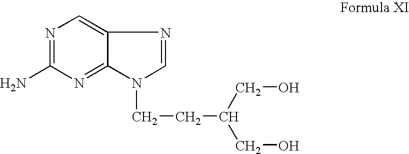
d) acylating the compound of Formula XI with a suitable acylating agent to give compound of Formula I.

15) The process according to claim 14, wherein the base employed is selected from triethylamine, sodium bicarbonate, sodium carbonate, potassium carbonate.

16) The process according to claim 14, wherein the solvent is ethanol.

17) The process according to claim 14, wherein the catalyst is selected from palladium, platinum, ruthenium, rhodium, raney nickel.

18) The process according to claim 14, wherein dechlorination is carried out in a solvent selected from C₁₋₆ alcohols.

19) The process according to claim 18, wherein the alcoholic solvent is selected from methanol, ethanol, isopropanol, n-butanol.

20) The process according to claim 14, wherein the dechlorination can also be carried out in the presence of transfer hydrogenation catalyst selected from ammonium formate, formic acid.

21) The process according to claim 14, wherein the deprotection is carried out using catalytic hydrogenation in presence of noble metal catalyst.

22) The process according to claim 21, wherein the noble metal catalyst is Palladium-carbon.

23) The process according to claim 14, wherein the deprotection can be carried out using an acid.

24) The process according to claim 23, wherein the acid is selected from hydrochloric acid, sulfuric acid, nitric acid.

25) The process according to claim 14, wherein the trialkylorthoformate employed is triethylorthoformate.

26) The process according to claim 14, wherein the acid employed is selected from hydrochloric acid, sulphuric acid, methanesulfonic acid, ethanesulfonic acid.

27) The process according to claim 14, wherein the acylating agent is acetyl chloride, acetic anhydride.

28) The process according to claim 14, wherein the compound of Formula II is prepared according to the process claimed in claim 1.

29) The process according to claim 14, optionally the intermediates are isolated or in situ used for further conversion.

30) A process for the preparation of Famciclovir of Formula I 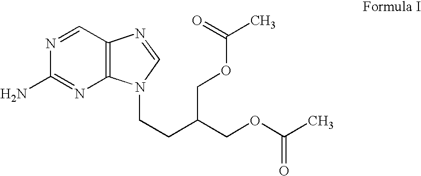
which comprises
a) deprotecting the compound of Formula II, 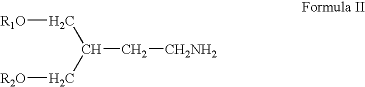
wherein R₁ and R₂ is same as defined above
to give compound of Formula VIII or a salt thereof 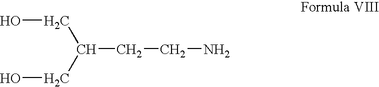
b) condensing the compound of Formula VIII or a salt thereof with a compound of Formula XII, 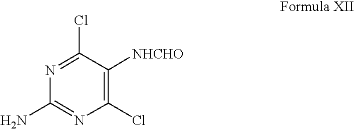
in presence of base in a solvent to give compound of Formula XIII, 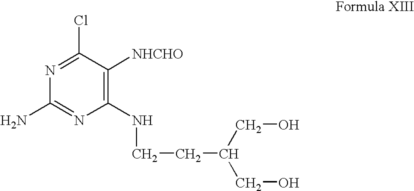
b) dechlorinating the compound of Formula XIII using a catalyst in a solvent to obtain a compound of Formula X, 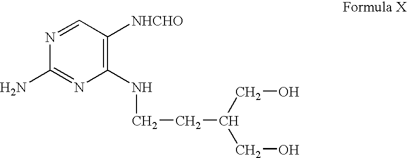
c) cyclizing the compound of Formula X with trialkylorthoformate in the presence of an acid to give compound of Formula XI, 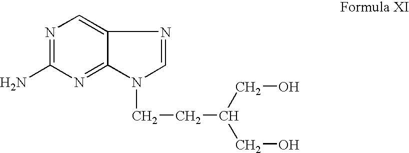
e) acylating the compound of Formula XI with a suitable acylating agent to give compound of Formula I.

31) The process according to claim 30, wherein the deprotection is carried out using catalytic hydrogenation in presence of noble metal catalyst.

32) The process according to claim 31, wherein the noble metal catalyst is Palladium-carbon.

33) The process according to claim 30, wherein the deprotection can be carried out using an acid.

34) The process according to claim 33, wherein the acid is selected from hydrochloric acid, sulfuric acid, nitric acid.

35) The process according to claim 34, wherein the base employed is selected from triethylamine, sodium bicarbonate, sodium carbonate, potassium carbonate.

36) The process according to claim 35, wherein the solvent is ethanol.

37) The process according to claim 30, wherein the catalyst is selected from palladium, platinum, ruthenium, rhodium, raney nickel.

38) The process according to claim 30, wherein dechlorination is carried out in a solvent selected from C₁₋₆ alcohols.

39) The process according to claim 30, wherein the alcoholic solvent is selected from methanol, ethanol, isopropanol, n-butanol.

40) The process according to claim 30, wherein the dechlorination can also be carried out in the presence of transfer hydrogenation catalyst selected from ammonium formate, formic acid.

41) The process according to claim 30, wherein the trialkylorthoformate employed is triethylorthoformate.

42) The process according to claim 30, wherein the acid employed is selected from hydrochloric acid, sulphuric acid, methanesulfonic acid, ethanesulfonic acid.

43) The process according to claim 30, wherein the acylating agent is acetyl chloride or acetic anhydride.

44) The process according to claim 30, wherein the compound of Formula II is prepared according to the process claimed in claim 1.

45) The process according to claim 30, wherein optionally the intermediates are isolated or in situ used for further conversion.
